(19) United States
(12) Patent Application Publication
(10) Pub. No.: US 2013/0310646 A1
(43) Pub. Date: Nov. 21, 2013

(54) DRIVING FORCE TRANSMISSION DEVICE FOR ENDOSCOPE AND AUXILIARY THRUST DEVICE COMPRISING THEREOF

(71) Applicant: FUJIFILM Corporation, Tokyo (JP)

(72) Inventors: Takumi DEJIMA, Ashigarakami-gun (JP); Tsuyoshi ASHIDA, Ashigarakami-gun (JP); Takayuki NAKAMURA, Ashigarakami-gun (JP); Nobuyuki TORISAWA, Ashigarakami-gun (JP); Naoyuki MORITA, Ashigarakami-gun (JP)

(73) Assignee: FUJIFILM Corporation, Tokyo (JP)

(21) Appl. No.: 13/893,975

(22) Filed: May 14, 2013

(30) Foreign Application Priority Data
May 15, 2012 (JP) 2012-111595

(51) Int. Cl. A61B 1/00; A61B 1/01

(52) U.S. Cl. CPC A61B 1/00154; A61B 1/0016; A61B 1/01
USPC 600/114

(57) ABSTRACT

An auxiliary thrust device includes a membrane, a carrier tube, and a drive cylinder. First and second insertion holes into which first and second torque wires are inserted are formed in a wire sheath. First and second gears are fixed to tip portions of the torque wires, which protrudes from a tip of the wire sheath, respectively. The first gear fixed to the first torque wire meshes with a spur gear portion of the drive cylinder, and the second gear connected to the second torque wire meshes with the first gear. Since the respective torque wires are inserted into one wire sheath and are bent in the same shape, a frictional force generated between the first torque wire and the inner surface of the wire sheath is substantially the same as that generated between the second torque wire and the inner surface of the wire sheath.

### BACKGROUND OF THE PRESENT INVENTION

1. Field of the Invention

The present invention relates to a driving force transmission device for an endoscope and an auxiliary thrust device of the endoscope that includes the driving force transmission device for the endoscope and thrusts a tip portion of the endoscope.

2. Description of the Related Art

Diagnosis using an endoscope is performed in a medical field. The endoscope includes an insertion portion that is inserted into a body, and an imaging element such as a CCD is built in a tip portion of the insertion portion. An image obtained by the imaging element is displayed in a monitor and an operator observes the inside of a body by the image that is displayed in the monitor.

An auxiliary thrust device of an endoscope that assists the thrust of an endoscope has been proposed in recent years. In WO2009/114137A2, a circulation body is mounted on a cylindrical support mounted on a tip portion of an endoscope so as to be capable of circulating, and a driving force is transmitted by a driving force transmission device for an endoscope that includes two drive shafts (which correspond to wires of the present invention) rotated by motors. The circulation body is circulated by the transmitted driving force while the outside of the circulation body comes into contact with the inside of a body, for example, the inner wall of an alimentary canal. Accordingly, the tip of the endoscope is self-propelled by the friction generated between the circulation body and the inner wall of an alimentary canal, and is guided into the alimentary canal. Accordingly, even a person who is unskilled at inserting an endoscope can easily insert the endoscope into the alimentary canal that is sinuous in the body, for example, a large intestine.

In WO2009/114137A2, a drive cylinder that is disposed in the circulation body is rotated by the rotation of rotating gears that are mounted on the tips of the two drive shafts, respectively. The circulation body, which is clamped between driving gears and rollers rotated by the rotation of the drive cylinder, is driven to circulate. Two bendable drive shafts are rotated in directions opposite to each other, the rotating gear mounted on the tip of one drive shaft meshes with a gear of the drive cylinder, and the rotating gear mounted on the tip of the other drive shaft meshes with one rotating gear. The two drive shafts are rotatably received in two sheaths, respectively, and the two sheaths are mounted on an insertion portion of an endoscope.

### SUMMARY OF THE PRESENT INVENTION

When the drive shafts are bent in the sheaths, in WO2009/114137A2, frictional forces are generated between the drive shafts and the inner surfaces of the sheaths due to the bending stiffness of the drive shafts and a rotational load is generated. Since the two sheaths are disposed independently of each other, the bending shapes of the two drive shafts are different from each other. In this case, since a difference in rotational load caused by the bending of both the drive shafts is generated, the drive shaft to which a larger rotational load is applied is prone to being damaged. Further, when strong load torque is applied to the drive shaft, torsional deformation occurs on the drive shaft and the drive shaft is deformed in a spiral shape in each sheath. Since the spiral shape of the deformed drive shaft is also changed according to the fluctuation of torque, the drive shaft rampages in the alimentary canal for each sheath. For this reason, there is a risk that the inner wall of the alimentary canal may be damaged.

The present invention has been made to solve the above-mentioned problem, and an object of the present invention is to provide a driving force transmission device for an endoscope that can improve safety and an auxiliary thrust device of an endoscope including the driving force transmission device.

In order to achieve the above-mentioned object, according to an aspect of the present invention, there is provided a driving force transmission device for an endoscope that includes bendable first and second wires, is mounted on an endoscope, transmits a driving force by the first and second wires rotated in directions opposite to each other by a wire drive unit, and drives a member related with the endoscope by the transmitted driving force. The driving force transmission device includes a wire sheath that includes a first insertion hole into which the first wire is inserted and a second insertion hole into which the second wire is inserted and receives the first and second wires so as to allow the first and second wires to rotate.

According to another aspect of the invention, there is provided an auxiliary thrust device of an endoscope including the driving force transmission device for an endoscope, a drive cylinder into which a tip portion of the endoscope is inserted and which is rotated by a driving force transmitted by the driving force transmission device for an endoscope, an outer tube that is disposed outside the drive cylinder, and a circulation body that is disposed so it is able to circulate in an axial direction of the drive cylinder while covering inner and outer peripheral surfaces of the outer tube, and circulates with the rotation of the drive cylinder.

In addition, it is preferable that the auxiliary thrust device further include a spur gear and a worm gear formed on an outer peripheral surface of the drive cylinder, a first gear meshing with the spur gear of the drive cylinder, and a second gear meshing with the first gear, the first wire be connected to the first gear, and the second wire be connected to the second gear.

It is preferable that the auxiliary thrust device of an endoscope further include a plurality of driving gears meshing with the worm gear of the drive cylinder and coming into contact with the circulation body to drive the circulation body.

It is preferable that the auxiliary thrust device of an endoscope further include a plurality of rollers rotatably mounted on the outer tube and pressing an inner peripheral surface of the circulation body to clamp the circulation body between the driving gears and the rollers while allowing the circulation body to rotate.

Moreover, it is preferable that the circulation body be formed of a rotating body formed in the shape of a bag so as to cover the outer tube over the entire circumference.

In addition, it is preferable that the circulation body be formed of a plurality of endless belts covering a part of the outer tube in a circumferential direction.

According to the present invention, since bendable first and second wires rotated in directions opposite to each other are received in one wire sheath, the first and second wires are bent in the same shape. Accordingly, when the first and second wires are bent, a frictional force generated between the first torque wire and an inner surface of the wire sheath is substantially the same as a frictional force generated between the second torque wire and the inner surface of the wire sheath. Therefore, a difference of a rotational load of a wire, which is generated due to a difference of a frictional force generated when the respective wires are bent in different shapes, is not generated and it is possible to prevent the damage of each of the wires that is caused by a difference of a rotational load.

### BRIEF DESCRIPTION OF THE DRAWINGS

FIG. 1 is a schematic view showing an endoscope on which an auxiliary thrust device of an endoscope is mounted.

FIG. 2 is a perspective view of the auxiliary thrust device of the endoscope.

FIG. 3 is an exploded perspective view of the auxiliary thrust device of the endoscope.

FIG. 4 is a perspective view showing gears, a drive cylinder, helical gears, and bearing rings.

FIG. 5 is an exploded perspective view showing first and second torque wires, a wire sheath, and first and second gears.

FIG. 6 is a perspective view showing the first and second torque wires, the wire sheath, and the first and second gears.

FIG. 7 is a side cross-sectional view of a portion of the helical gears.

FIG. 8 is a front view of a membrane.

FIG. 9 is a front cross-sectional view of a portion of the helical gears.

FIG. 10 is a perspective view showing the first and second torque wires, first and second cylindrical wire sheaths, and the first and second gears.

### DESCRIPTION OF THE PREFERRED EMBODIMENTS

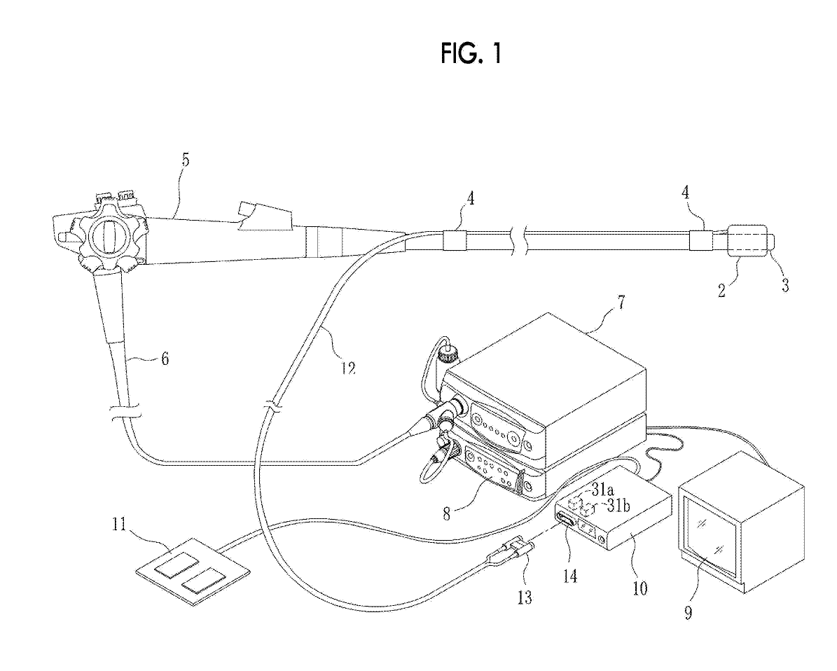

As shown in FIG. 1, an auxiliary thrust device 2 is fixed to a hard tip portion 3 of an endoscope when being used. For example, an imaging optical system and a CMOS or CCD image sensor are built in the hard tip portion 3, and take an image of a stomach wall or an intestinal wall with illumination light that is emitted from an illumination window provided at the hard tip portion 3. A bending portion is formed at the base end of the hard tip portion 3 so that the hard tip portion 3 can be inserted to a desired position. Further, the auxiliary thrust device 2 is used together in order to assist the insertion of the hard tip portion 3. The bending portion can be bent by the operation of an angle knob, which is provided on an operation portion 5, so as to be easily inserted.

The operation portion 5 is further provided with operation buttons that are used to switch the intake and exhaust of air and the suction and discharge of water, a cap of a forceps channel into which a biopsy forceps and the like are inserted, and the like. A connection cord 6 is led from the operation portion 5, and is connected to a light source device 7 and an endoscope processor 8. Light, which is emitted from an illumination lamp built in the light source device 7, is guided to the illumination window through a light guide fiber that is built in the connection cord 6 and the endoscope. The endoscope processor 8 appropriately processes an image signal input from the connection cord 6 and an obtained image is displayed in a display monitor 9. Meanwhile, the endoscope processor 8 can identify the information about the kind of the endoscope, which is currently connected to the endoscope processor 8, on the basis of information that is input from the endoscope through the connection cord 6. Further, when control different for each kind of an endoscope is needed at the time of the operation of the endoscope, when image display different for each kind of an endoscope is needed in the display monitor 9, or the like, it is possible to automatically switch control or display to appropriate control or display corresponding to the information about the kind of the endoscope.

A controller 10 is electrically connected to the endoscope processor 8. The controller 10 is used to monitor and control the operation of the auxiliary thrust device 2. A foot switch 11, which is used to start the drive of the auxiliary thrust device 2, is connected to the controller 10. The auxiliary thrust device 2 includes a flexible wire sheath 12 that is led from the rear end of the auxiliary thrust device 2 and is formed to be bifurcated. The wire sheath 12 is appropriately fixed to an insertion portion of the endoscope by a surgical tape 4 or the like. Accordingly, when the endoscope on which the auxiliary thrust device 2 has been mounted is inserted into and operated in a body cavity, the wire sheath 12 does not behave improperly in the body cavity.

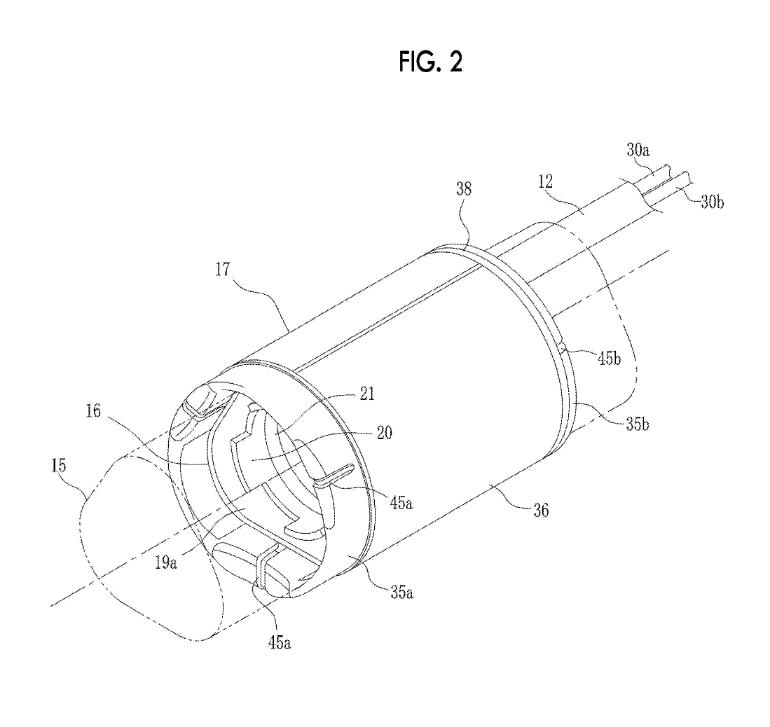

First and second torque wires 30a and 30b (see FIG. 2) are inserted into the double lumen-type wire sheath 12. The rear end portion of the wire sheath 12 is formed to be bifurcated, and the respective bifurcated portions of the rear end portion are connected to a bifurcated plug 13. The rear ends of the respective torque wires 30a and 30b are connected to a connector 14 of the controller 10 through the plug 13. Since a pair of first and second motors 31a and 31b are built in the controller 10, the respective torque wires 30a and 30b can be individually driven by the respective motors 31a and 31b when the plug 13 is connected to the connector 14.

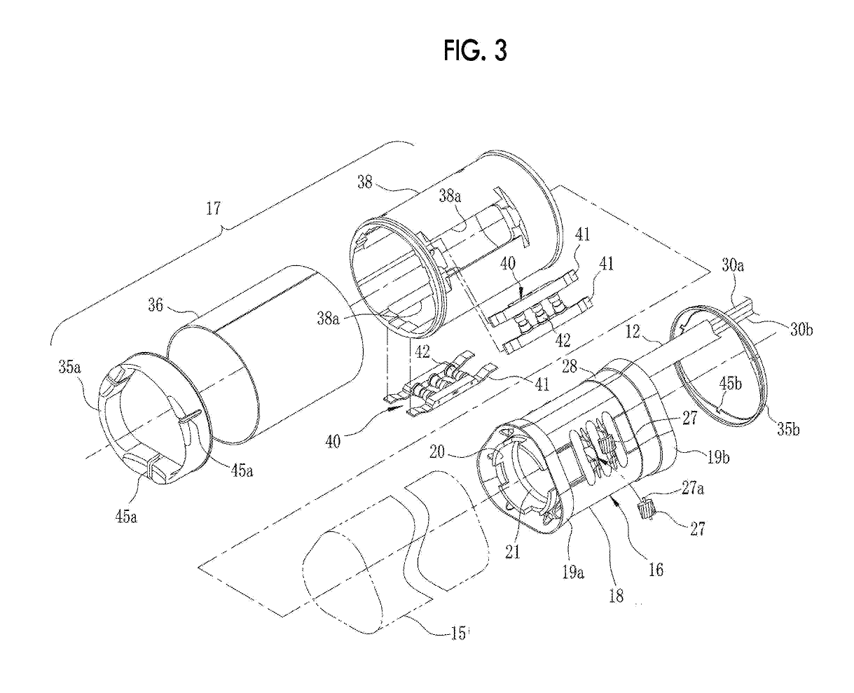

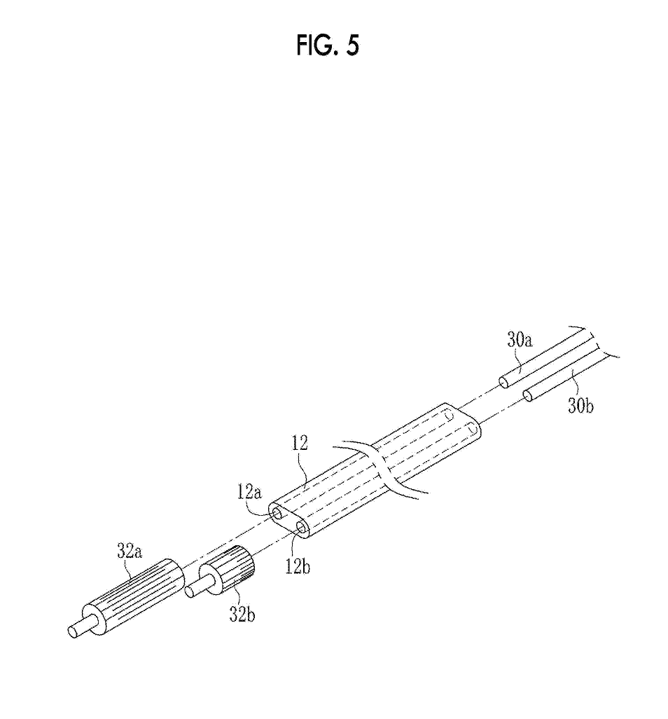

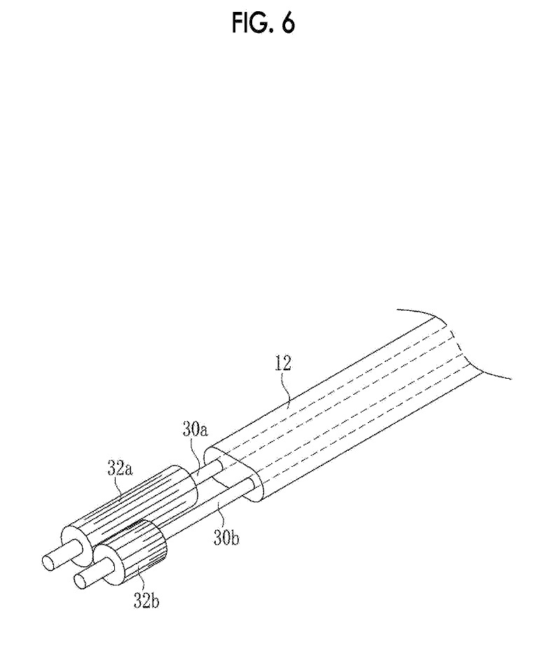

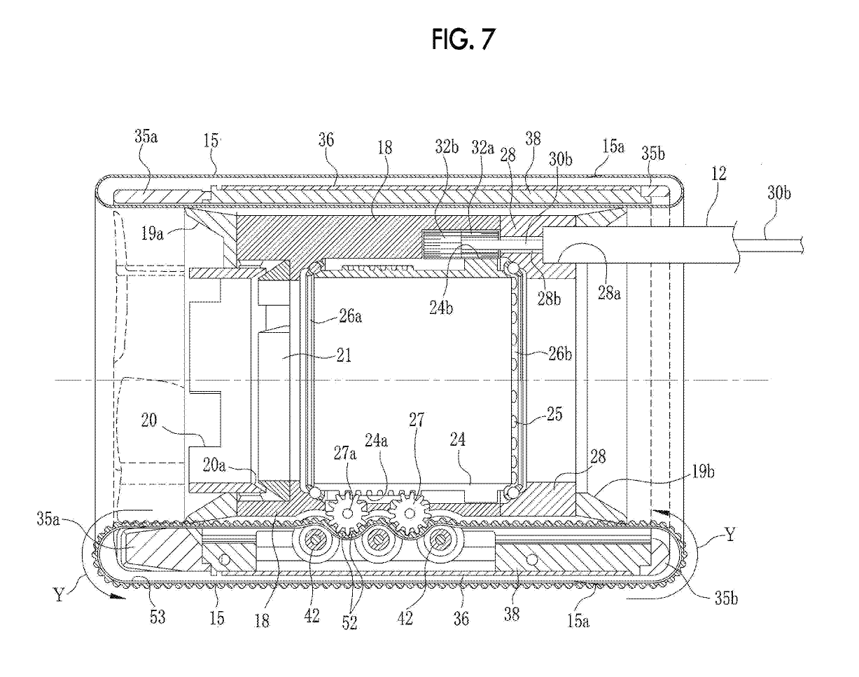

The auxiliary thrust device 2 is effectively used to easily insert or draw an endoscope for a large intestine into or out of, particularly, a sigmoid colon or a transverse colon. The auxiliary thrust device 2 has a substantially columnar shape, and the outer surface of the auxiliary thrust device 2 is covered with a membrane 15 formed of a sheet member that is a toroidal circulation moving body and is made of a flexible and strong synthetic resin. For the easy understanding of the structure, the membrane 15 is developed and shown in a cylindrical shape in FIGS. 2 and 3. However, the front end of the membrane 15 is joined to the rear end thereof while the membrane 15 is inverted so that the shown inner peripheral surface of the membrane 15 becomes an outer peripheral surface when the membrane 15 is finally built. As a result, the membrane 15 is formed in the shape of a toroidal bag body (see FIG. 7). Meanwhile, in FIGS. 2 to 7, the left side is a tip side toward the hard tip portion 3 protrudes and the right side is a base end side that is close to the operation portion 5 of the endoscope.

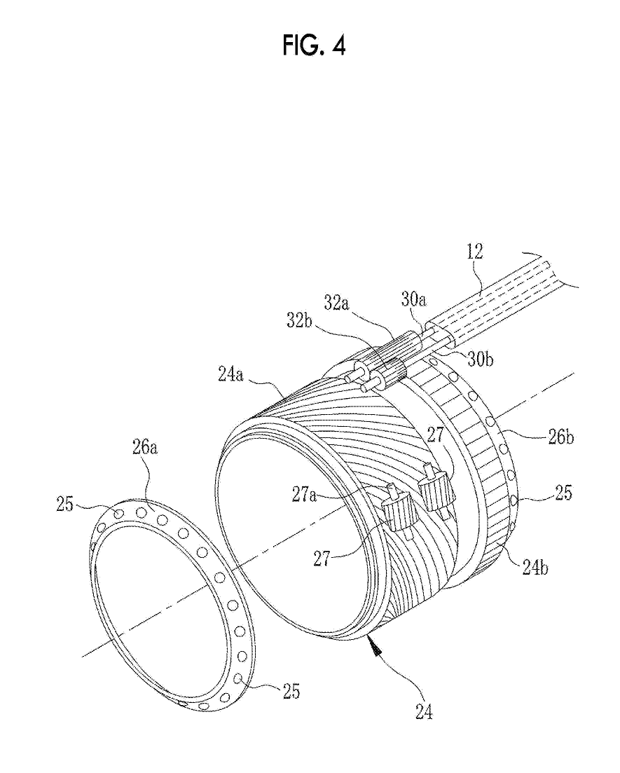

As shown in FIGS. 2 and 3, the auxiliary thrust device 2 includes an inner unit 16 that forms an inner structure of the membrane 15 developed into a cylindrical shape, and an outer unit 17 that forms an outer structure of the membrane 15. The inner unit 16 includes a carrier tube 18 which has a cylindrical hollow portion on the inner peripheral side thereof and of which the outer peripheral surface is formed in the shape of a triangular prism; a cap 28 that is locked to the rear end portion of the carrier tube 18 by screwing, press-fitting, calking, or the like and has substantially the shape of a triangular prism; front and rear wipers 19a and 19b that are fixed to the front end portion of the carrier tube 18 and the rear end portion of the cap 28, respectively; a damper 20 that is engaged with threads formed on the inner periphery of the front end portion of the carrier tube 18 and moves in an axial direction by rotation; a C-ring 21 which is made of a synthetic resin and of which the inner and outer diameters are increased/reduced according to the movement of the clamper 20 in the axial direction; and a cylindrical drive cylinder 24 (see FIG. 4) that is rotatably supported on the inner periphery of the carrier tube 18.

As shown in FIG. 4, both ends of the drive cylinder 24 are rotatably supported on the inner periphery of the carrier tube 18 with bearing rings 26a and 26b, which includes bearing balls 25 arranged and held in an annular shape, interposed therebetween and the drive cylinder 24 is retained by the cap 28 that is fixed to the rear end of the carrier tube 18. A worm gear portion 24a and a spur gear portion 24b are formed on the outer peripheral surface of the drive cylinder 24. Pairs of helical gears 27 and 27, which are rotatably held by the carrier tube 18, mesh with the worm gear portion 24a through openings of the carrier tube 18. The pairs of helical gears 27 and 27 are assembled at three positions that are symmetrically arranged around the rotation axis of the drive cylinder 24 at an interval of 120°. When the drive cylinder 24 is rotated, these helical gears 27 and 27 are rotated about shafts 27a all together in the same direction, respectively.

As shown in FIGS. 4 to 7, a first insertion hole 12a into which the first torque wire 30a is inserted and a second insertion hole 12b into which the second torque wire 30b is inserted are formed in the double lumen-type wire sheath 12 that is formed integrally. Tip portions of the torque wires 30a and 30b, which are inserted into the insertion holes 12a and 12b, respectively, protrude from the tip of the wire sheath 12. Each of the torque wires 30a and 30b is formed by winding a metal wire in the shape of a coil, has flexibility and torsional stiffness, and transmits torque, which is input to the rear end portion from each of the motors 31a and 3 lb of the controller 10, to the tip side without reducing most of the torque.

The bending stiffness of the wire sheath 12 is higher than the bending stiffness of each of the torque wires 30a and 30b. Since the sheath 12 is formed to be wider than a cylindrical sheath into which one torque wire is inserted so that the torque wires 30a and 30b are inserted into the wire sheath 12, the thickness of the sheath 12 is the same as the thickness of the cylindrical sheath but the bending stiffness of the sheath 12 is higher than that of the cylindrical sheath. Accordingly, it is possible to make the bending stiffness of the wire sheath 12 higher than the bending stiffness of each of the torque wires 30a and 30b without an increase in thickness.

The tip of the wire sheath 12 is fixed to a mounting recess 28a, which is formed in the cap 28, by adhesion, thermal welding, or the like. Further, the tip portions of the torque wires 30a and 30b, which protrude from the tip of the wire sheath 12, protrude forward from the cap 28 through a through hole 28b that is formed in the cap 28, and first and second gears 32a and 32b are fixed to the tip portions of the torque wires 30a and 30b, respectively.

A shaft, which corresponds to the center of rotation, protrudes from the tip of each of the gears 32a and 32b. These shafts are inserted into holes formed in the carrier tube 18, so that each of the gears 32a and 32b is rotatably supported.

Among the gears 32a and 32b, the first gear 32a fixed to the first torque wire 30a meshes with the spur gear portion 24b of the drive cylinder 24. The second gear 32b connected to the other second torque wire 30b meshes with the first gear 32a and does not mesh with the spur gear portion 24b. Accordingly, the drive cylinder 24 is driven by the rotation of the first gear 32a that is connected to the first torque wire 30a. However, the torque wires 30a and 30b are driven by the torque individually transmitted from the controller 10, and the second gear 32b is rotated in the direction opposite to the rotation direction of the first gear 32a. For this reason, the torque transmitted from the second torque wire 30b is also applied to the first gear 32a, so that it is possible to rotate the drive cylinder 24 by high torque.

Each of the front and rear wipers 19a and 19b includes a sleeve portion, which becomes wider in the shape of an eave, at the tip portion thereof, and the tips of these sleeve portions come into sliding contact with the inner periphery of the membrane 15 when the membrane 15 moves to circulate.

Further, the front and rear wipers 19a and 19b prevent foreign materials, which adhere to the inner peripheral surface of the membrane 15, or the inner wall of an alimentary canal from being drawn into the auxiliary thrust device 2 with the movement of the membrane 15.

Since regular uneven engaging portions are formed at the front end of the damper 20 so as to be aligned in the circumferential direction, a dedicated jig can be inserted from the tip side and engaged with the clamper 20. When the damper 20 is rotated in a screwing direction by the rotation of the jig, the damper 20 is moved rearward and the C-ring 21 is pressed by an inclined surface 20a formed at the rear end. Accordingly, the C-ring 21 is deformed so that the diameter of the C-ring 21 is reduced. Therefore, when the screwing of the damper 20 is performed after the hard tip portion 3 of the endoscope is inserted into the cylindrical hollow portion of the carrier tube 18, the inner peripheral surface of the C-ring 21 is strongly pressed against the outer peripheral surface of the hard tip portion 3. As a result, it is possible to fix the carrier tube 18 to the hard tip portion 3.

The outer unit 17, which forms an outer structure of the membrane 15, includes a front bumper 35a, a shield cover 36, a cylindrical holding tube (outer tube) 38, and a rear bumper 35b that are positioned in this order from the tip side. The outer unit 17 is assembled so as to be integrally connected to the inner unit 16 and the membrane 15 according to the following procedure.

After the inner unit 16 is put into the membrane 15 developed into a cylindrical shape so that the outer surface of the inner unit 16 in which various components have been built is covered as shown in FIGS. 2 and 3, the inner unit 16 covered with the membrane 15 is inserted into a hollow portion of the holding tube 38. Substantially rectangular openings 38a, which are long in the axial direction, are formed on the holding tube 38 at three positions that are symmetrically arranged around the axis of the holding tube 38 at an interval of 120°. Roller assemblies 40 are assembled in the openings 38a.

As shown in FIG. 3, the roller assembly 40 includes three rollers 42 that are aligned and held between a pair of elongate frames 41. The frame 41 is formed of an elastic thin metal plate. When both ends of the frame 41 are fitted into engaging portions formed at the front and rear ends of the opening 38a and are fixed to the holding tube 38, the frame 41 is bent so that the middle portion of the frame 41 in the longitudinal direction enters the hollow portion of the holding tube 38 through the opening 38a. Since the frames 41 are bent in this way, the three rollers 42 held by the frames 41 press the membrane 15 against the helical gears 27. As a result, as shown in FIG. 7, the membrane 15 is strongly clamped between the pair of helical gears 27 and the three rollers 42. Here, since a support shaft of the intermediate roller 42 among the three rollers is supported in long holes of the frames 41 that extend in the longitudinal direction of the frame 41, the intermediate roller 42 has a degree of freedom in position in the longitudinal direction of the frame 41. Accordingly, the position of the intermediate roller 42 relative to the two rollers positioned on both sides is automatically adjusted to a position where the membrane 15 can be clamped with the best balance between the pair of helical gears 27.

When the roller assemblies 40 are assembled on the holding tube 38 so as to cover the three openings 38a in this way, the respective rollers 42 protrude into the holding tube 38. Accordingly, the holding tube 38 cannot move relative to the inner unit 16 in the axial direction, so that the holding tube 38 and the inner unit 16 are integrally combined with each other with the membrane 15 interposed therebetween. Further, the front bumper 35a is fixed to at the front end of the holding tube 38 and the rear bumper 35b is fixed to the rear end of the holding tube 38. Grooves 45a and 45b are formed at three positions on the front end portion of the front bumper 35a and the rear end portion of the rear bumper 35b so as to be aligned with the roller assemblies 40 in the axial direction, respectively. In addition, the outer surface of the holding tube 38 and the roller assemblies 40 are closely covered with the shield cover 36.

After the membrane 15, which is developed into a cylindrical shape, is interposed between the inner and outer units 16 and 17 as shown in FIG. 7 and these are integrally combined with each other, the front and rear ends of the membrane 15 are inverted in the opposite direction and then joined to each other. In this case, if the respective joint surfaces are formed to be inclined, extremely uneven thickness does not occur at a joint 15a. FIG. 7 schematically shows the cross-section of the auxiliary thrust device 2 that has been assembled. The membrane 15 is joined in a toroidal shape as described above, so that the membrane 15 has an interior space wrapping the outer unit 17 as a whole. An appropriate material, for example, lubricant, such as air, physiological saline, a colloidal synthetic resin material, oil, or grease, may be sealed in this interior space.

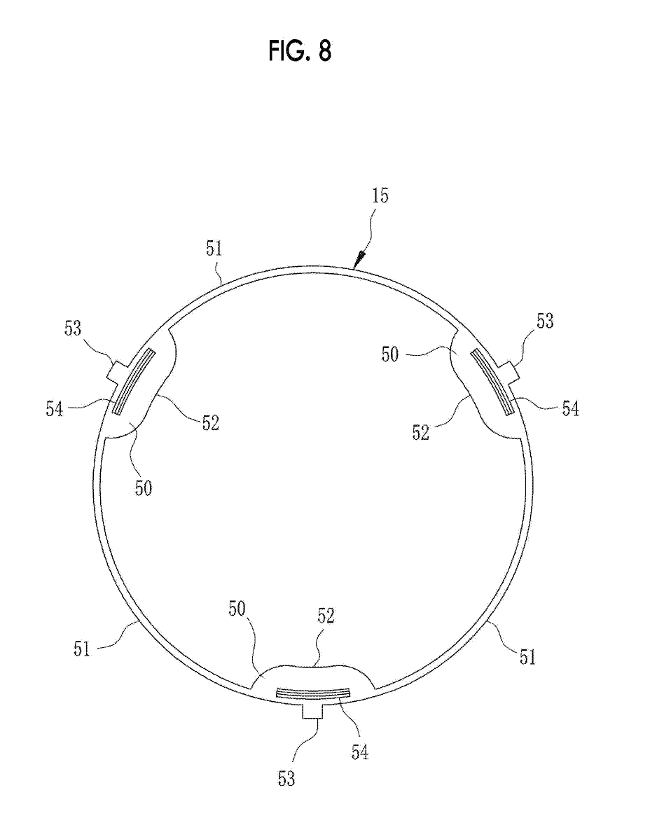

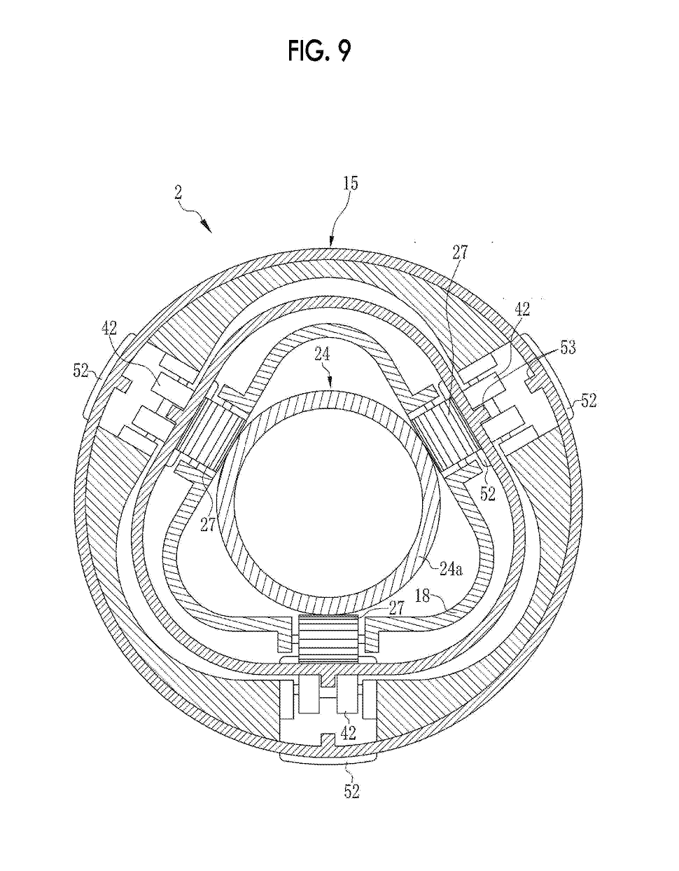

As shown in FIGS. 7 to 9, the membrane 15 has a structure where sheet materials such as an elastically deformable urethane resin are laminated, and protruding portions 50 having a trapezoidal cross-section are formed on the inner periphery of the membrane 15 at the positions where circumference is divided into three equal pieces. The protruding portion 50 is a portion that is formed to be thick by increasing the number of sheet materials to be laminated as compared to other thin portions 51, and is formed over the entire length of the membrane 15 in the axial direction. A plurality of gear teeth 52, which are inclined so as to mesh with the helical gear 27, are formed on the protruding portion 50 at a predetermined pitch.

Further, ribs 53, which extend in the axial direction, are formed on the outer peripheral surface of the membrane 15 so as to correspond to the positions of the protruding portions 50, and mesh-shaped fiber sheets 54 are laminated between the gear teeth 52 and the rib 53.

When the membrane 15 is used, both sheet-shaped end portions of the membrane 15 are joined to each other so that the membrane 15 is formed in a toroidal shape as shown in FIG. 7. In this case, each of the three protruding portions 50, which extend in the axial direction, is clamped between the helical gears 27 and rollers 42 and the helical gears 27 mesh with the gear teeth 52. Further, since the rotation of the helical gears 27 is directly transmitted to the membrane 15 through the gear teeth 52, it is possible to efficiently move the membrane 15 in the axial direction. Since the protruding portion 50 has a multi-layered structure of sheet materials and the mesh-shaped fiber sheets 54 are laminated, the membrane 15 can ensure sufficient mechanical strength without being fractured even though a driving force is directly applied to the protruding portion 50 from the helical gears 27. Furthermore, since other portions of the membrane 15 except for the protruding portions 50 are formed of thin portions 51, it is possible to reduce resistance when the membrane 15 passes between the inner and outer units 16 and 17.

In addition, as the membrane 15 is moved, the ribs 53, which are formed on the inner surfaces of the protruding portions 50, are engaged with grooves formed on the middle portions of the rollers 42. Further, when the toroidal interior space is adjusted to be reduced in size so that the outer unit 17 is closely wrapped with the membrane 15, the ribs 53 are engaged with even the grooves 45a and 45b (see FIG. 2) of the front and rear bumpers 35a and 35b. It is possible to prevent the skew of the membrane 15, which occurs when the membrane 15 is moved in the axial direction, and to maintain the moving path stable by using the ribs 53 as described above.

Next, the action of the auxiliary thrust device 2 will be described. As shown in FIG. 1, the auxiliary thrust device 2 is fixed to the endoscope so that the tip of the hard tip portion 3 partially protrudes from the auxiliary thrust device 2. When the auxiliary thrust device 2 is fixed to the endoscope, a dedicated jig is used to rotate the damper 20 in a clockwise direction. Since the clamper 20 is engaged with right-handed threads that are formed on the inner periphery of the front end portion of the carrier tube 18, the damper 20 is moved to the back side (rear end side) by being rotated in the clockwise direction and presses the C-ring 21 by the inclined surface 20a. An inclined surface, which is inclined rearward toward the outer periphery, on the front surface of the C-ring 21 and the inclined surface is pressed by the inclined surface 20a of the clamper 20, so that the C-ring 21 is elastically deformed so that the diameter of the C-ring 21 is reduced. When the C-ring 21 is deformed as described above, the hard tip portion 3 of the endoscope inserted into the hollow portion of the carrier tube 18 is tightened by the C-ring 21. Accordingly, the auxiliary thrust device 2 is closely fixed to the outer peripheral surface of the hard tip portion 3.

The wire sheath 12, which is led from the rear end of the auxiliary thrust device 2, is stretched out from the bending portion of the endoscope along the surface of a flexible portion. Marks, which indicate tape retaining positions, are formed on the surface of the wire sheath 12 at appropriate intervals. The wire sheath 12 is fixed to the bending portion or the flexible portion of the endoscope with a surgical tape 4 or the like in accordance with the marks. Further, the plug 13 provided at the rear end of the sheath is inserted into the connector 14 and is connected to be controller 10, so that electric power of the controller 10 is turned on. When the electric power of the controller 10 is turned on, the controller 10 electrically checks whether or not the plug 13 is connected to the connector 14. If the plug 13 is not connected to the connector 14 or is not properly connected to the connector 14, the controller 10 notifies a user of the non-connection or improper connection of the plug 13 with sound, the flicker of a warning lamp, or the like. If the plug 13 is properly connected to the connector 14, a sensor built in the connector 14 reads out information about the kind of the auxiliary thrust device 2 from a signal part provided on a bridge portion of the plug 13. Further, the controller 10 automatically sets the rotational speeds of the torque wires or a value of a torque limiter according to the information about the kind of the auxiliary thrust device 2 that have been read out, and prevents the torque wires 30a and 30b from being rotated with excessive speed or torque.

Furthermore, when the electric power of the controller 10 is turned on, the controller 10 receives information about the kind of the endoscope, which is connected to the endoscope processor 8, as electrical signals that are sent from the endoscope processor 8. The controller 10 collate information about the kind of the currently-used endoscope and information about the kind of the auxiliary thrust device 2 with table information that is stored in internal storage means of the controller 10. Collation data, which correspond to information about the kind of the auxiliary thrust device 2 that can be applied for each kind of an endoscope, are stored in the table information. Moreover, for example, if a range where the diameter of the C-ring 21 is increased or reduced is specified from the information about the kind of the auxiliary thrust device 2 and the outer diameter of the hard tip portion 3 of the endoscope is specified from the information about the kind of the endoscope, it is possible to instantly determine whether or not the auxiliary thrust device 2 can be used while being properly mounted on the hard tip portion 3 of the endoscope. Accordingly, if it is determined that the combination of the auxiliary thrust device 2 and the endoscope is not proper, it is possible to prevent the occurrence of an unexpected accident by performing notification with warning sound, the flicker of a warning lamp, or the like or taking measures for preventing the operation of the auxiliary thrust device 2 or the like.

When the foot switch 11 connected to the controller 10 is operated, the pair of first and second motors 31a and 31b are driven in the controller 10 and torque is applied to the first and second torque wires 30a and 30b. The first torque wire 30a is rotated in the direction opposite to the rotation direction of the second torque wire 30b. Since the respective torque wires 30a and 30b are inserted into one wire sheath 12, the torque wires 30a and 30b are bent in the same shape. Accordingly, when the torque wires 30a and 30b are bent, a frictional force generated between the torque wire 30a and the inner surface of the wire sheath 12 is substantially the same as a frictional force generated between the torque wire 30b and the inner surface of the wire sheath 12. Therefore, the damage of each of the torque wires 30a and 30b, which is caused by a difference of a rotational load generated due to a difference of a frictional force generated when the respective torque wires 30a and 30b are bent in different shapes, is prevented.

Further, since the first and second torque wires 30a and 30b are rotated in directions opposite to each other, a first force that deforms the wire sheath 12 by the rotation of the first torque wire 30a and a second force that deforms the wire sheath 12 by the rotation of the second torque wire 30b are forces opposite to each other. Furthermore, since the first and second forces offset each other, so that a force for deforming the wire sheath 12 is reduced, the deformation of the wire sheath 12 is suppressed as compared to a case where the respective torque wires 30a and 30b are rotated in the same direction. Accordingly, the damage of the intestinal wall, which is caused by significant deformation (for example, spiral deformation) of the wire sheath 12, is prevented.

The first and second gears 32a and 32b are rotated by the rotation of the respective torque wires 30a and 30b, so that the drive cylinder 24 is rotated by the spur gear portion 24b meshing with the first gear 32a. The second gear 32b is rotated in the direction opposite to the rotation direction of the first gear 32a, and the rotation of the second gear 32b is transmitted to the first gear 32a without change. Accordingly, it is possible to rotate the drive cylinder 24 by using both the pair of first and second motors 31a and 31b built in the controller 10.

When the worm gear portion 24a is rotated together with the rotation of the drive cylinder 24, the helical gears 27 are rotated about the shafts 27a all together in the same direction, respectively. The membrane 15 is strongly clamped between the tooth surfaces of the helical gears 27 and the respective rollers 42 of the roller assemblies 40 and the helical gears 27 mesh with the gear teeth 52 of the membrane 15. Accordingly, the membrane 15, which is clamped between the helical gears 27 and the rollers 42 driven together with the rotation of the helical gears 27, is moved in the axial direction of the drive cylinder 24. For example, if the helical gears 27 are rotated in the clockwise direction in FIG. 7, the rollers 42 are rotated in the counterclockwise direction and the membrane 15 clamped between the helical gears 27 and the rollers 42 is fed to be moved to the tip side from the rear end side on the inner peripheral side (the inside of the outer unit 17) and is fed to the rear end side from the tip side on the outer peripheral side of the membrane 15 (the outside of the outer unit 17). That is, as shown in FIG. 7 by an arrow Y, the toroidal membrane 15 moves to circulate on the entire circumference of the toroidal membrane 15 so as to be sequentially fed to the outer peripheral side from the inner peripheral side at the front end and so as to be sequentially fed to the inner peripheral side from the outer peripheral side at the rear end.

When the endoscope and the auxiliary thrust device 2 are inserted into a large intestine and the outer peripheral surface of the membrane 15 comes into contact with an intestinal wall, while the membrane 15 moves to circulate as described above, a thrust force in the direction where the hard tip portion 3 of the endoscope is moved forward is obtained or a force for pulling the wall of the large intestine to the front side can be obtained.

While the membrane 15 is moved, foreign materials or the like adhering to the outer peripheral surface of the membrane 15 are moved to the inner peripheral side from the rear end side of the outer unit 17. However, immediately before the foreign materials are moved to the inner peripheral side, the tip of the sleeve portion extending toward the rear end side of the rear wiper 19b comes into sliding contact with the membrane 15 and prevents the foreign materials from being drawn. Of course, it is also possible to prevent a part of body tissue from being rolled together with the movement of the membrane 15. Meanwhile, when the membrane 15 moves to circulate in a reverse direction, the tip of the sleeve portion of the front wiper 19a functions in the same way as described above.

Light emitted from the light source device 7 is irradiated on the inside of the large intestine through the connection cord 6, the light guide fiber built in the endoscope, and the illumination window. A CCD built in the hard tip portion 3 radiographs the inside of an alimentary canal and outputs an imaging signal. This imaging signal is input to the endoscope processor 8 through a signal output cable built in the endoscope and the connection cord 6, and an image is displayed in the display monitor 9. An operator observes the inside of the alimentary canal through the display monitor 9.

If the operator finds out an affected part during the observation, the operator inserts a treatment tool suitable for a treatment of the affected part into a forceps inlet of the endoscope so that the treatment tool protrudes from a forceps outlet (not shown) and treats the affected part.

In order to remove the endoscope by moving the endoscope back, the operator rotates the pair of first and second motors 31a and 31b in the reverse direction and also rotates the first and second torque wires 30a and 30b in the reverse direction in the controller 10 by operating the foot switch 11. Accordingly, the membrane 15 is circulated in the direction opposite to the direction of the arrow Y of FIG. 7 and the endoscope is removed. Since the first and second torque wires 30a and 30b are rotated in directions opposite to each other even in this case, the deformation of the wire sheath 12 is suppressed.

In order to remove the auxiliary thrust device 2 from the hard tip portion 3, the operator rotates the damper 20 in the counterclockwise direction using a jig. Accordingly, the damper 20 moves forward while rotating, so that the pressing of the C-ring 21 is released. As a result, since the diameter of the C-ring 21 is increased due to its own elasticity and the inner peripheral surface of the C-ring 21 is separated from the outer peripheral surface of the hard tip portion 3, it is possible to easily remove the auxiliary thrust device 2 from the endoscope.

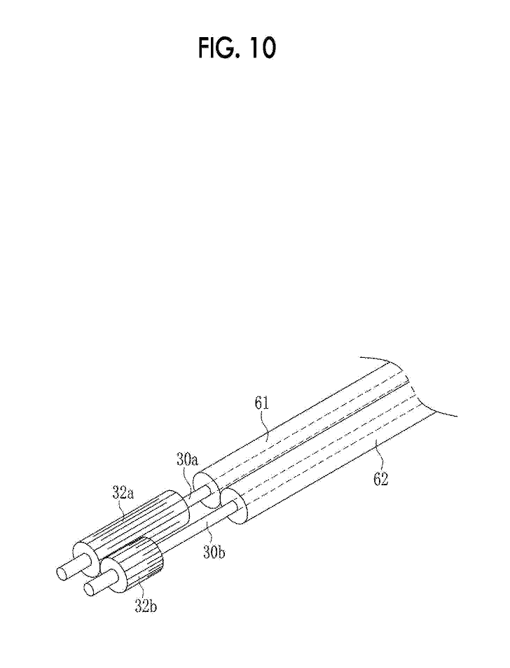

Meanwhile, the integrally formed double lumen-type wire sheath has been used in the embodiment, but two first and second cylindrical wire sheaths 61 and 62, which are joined to each other as shown in FIG. 10, may be used.

Further, the membrane has been driven to circulate by the helical gears meshing with the worm gear portion of the drive cylinder in the embodiment, but the worm gear portion of the drive cylinder may be pressed against the membrane to circulate the membrane without the helical gears.

Furthermore, the present invention has been applied to the auxiliary thrust device of an endoscope that assists the forward and backward movement of the endoscope in the embodiment, but the present invention may be applied to an auxiliary device that assists at least the forward movement of the endoscope.

Moreover, the forward and backward movement of the endoscope has been assisted by a rotating body that covers the holding tube over the entire circumference in the embodiment, but the forward and backward movement of the endoscope may be assisted by a plurality of endless belts that cover a part of the holding tube in the circumferential direction.

In addition, the present invention has been applied to an endoscope for medical diagnosis in the embodiment, but the present invention is not limited to a use for medical diagnosis and may be applied to other endoscopes such as an industrial endoscope, probes, and the like.

Further, a circulation body, which assists the thrust of the endoscope, has been driven by the driving force transmitted by the wires in the embodiment, but the present invention may be applied to any structure as long as a member related with an endoscope is driven by a driving force that is transmitted through the rotation of two wires in directions opposite to each other.

What is claimed is:

1. A driving force transmission device for an endoscope that includes bendable first and second wires, is mounted on the endoscope, transmits a driving force by the first and second wires rotated in directions opposite to each other by a wire drive unit, and drives a member related with the endoscope by the transmitted driving force, the driving force transmission device comprising:
a wire sheath that includes a first insertion hole into which the first wire is inserted and a second insertion hole into which the second wire is inserted and receives the first and second wires so as to allow the first and second wires to rotate.

2. An auxiliary thrust device of an endoscope comprising:
the driving force transmission device for the endoscope according to claim 1;
a drive cylinder into which a tip portion of the endoscope is inserted and which is rotated by the driving force transmitted by the driving force transmission device for the endoscope;
an outer tube that is disposed outside the drive cylinder; and
a circulation body that is disposed so as to circulate in an axial direction of the drive cylinder while covering inner and outer peripheral surfaces of the outer tube, and circulates with the rotation of the drive cylinder.

3. The auxiliary thrust device of the endoscope according to claim 2, further comprising:
a spur gear and a worm gear that are formed on an outer peripheral surface of the drive cylinder;
a first gear that meshes with the spur gear of the drive cylinder; and
a second gear that meshes with the first gear,
wherein the first wire is connected to the first gear, and
the second wire is connected to the second gear.

4. The auxiliary thrust device of the endoscope according to claim 3, further comprising:
a plurality of driving gears that mesh with the worm gear of the drive cylinder and come into contact with the circulation body to drive the circulation body.

5. The auxiliary thrust device of the endoscope according to claim 4, further comprising:
a plurality of rollers that are rotatably mounted on the outer tube and press an inner peripheral surface of the circulation body to clamp the circulation body between the driving gears and the rollers while allowing the circulation body to rotate.

6. The auxiliary thrust device of the endoscope according to claim 2,
wherein the circulation body is formed of a rotating body that is formed in the shape of a bag so as to cover the outer tube over the entire circumference.

7. The auxiliary thrust device of the endoscope according to claim 3,
wherein the circulation body is formed of a rotating body that is formed in the shape of a bag so as to cover the outer tube over the entire circumference.

8. The auxiliary thrust device of the endoscope according to claim 4,
wherein the circulation body is formed of a rotating body that is formed in the shape of a bag so as to cover the outer tube over the entire circumference.

9. The auxiliary thrust device of the endoscope according to claim 5,
wherein the circulation body is formed of a rotating body that is formed in the shape of a bag so as to cover the outer tube over the entire circumference.

10. The auxiliary thrust device of the endoscope according to claim 2,
wherein the circulation body is formed of a plurality of endless belts that cover a part of the outer tube in a circumferential direction.

11. The auxiliary thrust device of the endoscope according to claim 3,
wherein the circulation body is formed of a plurality of endless belts that cover a part of the outer tube in a circumferential direction.

12. The auxiliary thrust device of the endoscope according to claim 4,
wherein the circulation body is formed of a plurality of endless belts that cover a part of the outer tube in a circumferential direction.

13. The auxiliary thrust device of the endoscope according to claim 5,
wherein the circulation body is formed of a plurality of endless belts that cover a part of the outer tube in a circumferential direction.
